(19) United States
(12) Patent Application Publication
(10) Pub. No.: US 2016/0051829 A1
(43) Pub. Date: Feb. 25, 2016

(54) MULTIFUNCTIONAL DEVICE FOR THE TREATMENT OF HEADACHE AND MOOD DISORDERS

(71) Applicant: Amir Porat, Savyon (IL)

(72) Inventor: Amir Porat, Savyon (IL)

(21) Appl. No.: 14/306,707

(22) Filed: Jun. 17, 2014

(51) Int. Cl. A61N 5/06; A61M 35/00; A61M 21/02

(52) U.S. Cl. CPC A61N 5/062; A61M 21/02; A61M 35/003; A61M 2202/0216; A61N 2005/0651; A61M 2037/0007

(57) ABSTRACT

The disclosure is directed in general to a portable device for the non-invasive treatment of headaches and mood disorders. Specifically, the disclosure is directed to a portable device capable of simultaneously and selectively generate at least three synergistic modes of treatment of headaches and mood disorders at varying intensities and durations.

### BACKGROUND

The present disclosure relates in general to a multifunctional device for the non-invasive treatment of headaches and mood disorders. Specifically, the disclosure relates to a portable device capable of simultaneously and selectively generating at least three synergistic modes operation, creating a controlled atmosphere around an organ of a subject in need thereof.

Headache pain is highly prevalent amongst people worldwide. The World Health Organization estimates that one person in 20 has a headache every day or almost every day. In addition, approximately 70% of adults in developed countries are afflicted with tension or “stress” headaches. The World Health Organization estimates that 240 million people worldwide each year are afflicted with migraine headaches. Headache pain can impose disabling hardships on afflicted individuals such as personal suffering, impaired quality of life, and impaired financial status. Headache pain, in some instances together with the constant fear of such pain, can damage an afflicted individual's family life, social life, workplace productivity, etc. Moreover, individuals who suffer long-term chronic headache pain can be predisposed to suffer other illnesses; for example, depression is three times more common in people afflicted with recurring migraine or severe headaches than in people who do not suffer from recurring migraine or severe headaches.

It has also been suggested that depressive disorders exist on a continuum, with non-clinical symptoms causing considerable burden to the population, increasing individual risk of developing major depressive disorder. An alternative strategy to professional treatment of non-clinically qualifying depression can be the promotion of effective self-help interventions that can be easily self-applied.

Therefore there is a need for a device that provides effective, self-administered treatment to headaches of various types, as well as non-clinically qualifying mood disorders and other circumstances where synergistic effect of various operation modes can provide health benefit.

### SUMMARY

In an embodiment, provided herein is a portable device for treating, for example, a mood disorder, a headache or their combination in a subject in need thereof, the device comprising: a substantially elongated housing, the housing comprising: a dorsal housing portion and a ventral housing portion, each housing portion having a proximal end and a distal end; an air inlet opening disposed in each of the dorsal housing portion and the ventral housing portion; at least one air outlet slot disposed in the ventral housing portion and located between the ventral air inlet opening and the distal end of the ventral portion; a housing cap, operably coupled to the distal end of each of the dorsal housing portion and the ventral housing portion, the housing cap comprising, for example an interchangeable lens and defines a plurality of arcuate slits in the periphery of the lens, wherein the dorsal housing portion is operably coupled to the ventral housing portion so as to define at least one compartment between the dorsal housing portion and the ventral housing portion; and a display panel coupled to the dorsal housing portion; a power supply; a negative ion generating module; a phototherapeutic module; a vibration generator; a control panel; and optionally an ozone module, wherein the negative ion module, phototherapeutic module, the vibration generator and optionally the ozone generator being in electronic communication with the power supply and the control panel, and wherein the device, using the control panel, is configured to selectively release negative ions through the slits disposed in the housing cap and/or the at least one air outlet slot in the ventral housing portion, and/or vibrate the housing cap, and/or emit phototherapeutic electromagnetic radiation through the lens.

In another embodiment, provided herein is a method of treating a mood disorder, a headache or their combination in a subject in need thereof, comprising: providing a device comprising; a substantially elongated housing, the housing comprising: a dorsal housing portion and a ventral housing portion, each housing portion having a proximal end and a distal end; an air inlet opening disposed in each of the dorsal housing portion and the ventral housing portion; at least one air outlet slot disposed in the ventral housing portion and located between the ventral air inlet opening and the distal end of the ventral portion; a housing cap, operably coupled to the distal end of each of the dorsal housing portion and the ventral housing portion, the housing cap comprising, for example a lens and defines a plurality of arcuate slits in the periphery of the lens, wherein the dorsal housing portion is operably coupled to the ventral housing portion so as to define at least one compartment between the dorsal housing portion and the ventral housing portion; and a display panel coupled to the dorsal housing portion; a power supply; a negative ion module; a phototherapeutic module; a vibration generator; a control panel; and optionally an ozone module, wherein the negative ion module, phototherapeutic module, the vibration generator and optionally the ozone generator in electronic communication with the power supply and the control panel, wherein the device, using the control panel, is configured to selectively release negative ions through the slits disposed in the housing cap and/or the at least one air outlet slot in the ventral housing portion, and/or vibrate the housing cap, and/or emit phototherapeutic electromagnetic radiation through the lens; placing the distal end of the device abutting the forehead of the subject; and using the control panel, selectively releasing negative ions through the slits disposed in the housing cap or the at least one air outlet slot in the ventral housing portion, vibrating the housing cap, emitting phototherapeutic electromagnetic radiation through the lens, optionally generating ozone, or effecting any combination of at least three of the foregoing.

In yet another embodiment, provided herein is a method of increasing the permeability of at least a portion of the skin, comprising: providing a device comprising; a substantially elongated housing, the housing comprising: a dorsal housing portion and a ventral housing portion, each housing portion having a proximal end and a distal end; an air inlet opening disposed in each of the dorsal housing portion and the ventral housing portion; at least one air outlet slot disposed in the ventral housing portion and located between the ventral air inlet opening and the distal end of the ventral portion; a housing cap, operably coupled to the distal end of each of the dorsal housing portion and the ventral housing portion, the housing cap comprising an interchangeable lens and defines a plurality of arcuate slits in the periphery of the lens, wherein the dorsal housing portion is operably coupled to the ventral housing portion so as to define at least one compartment between the dorsal housing portion and the ventral housing portion; a power supply; and a display panel coupled to the dorsal housing portion; a negative ion module; a phototherapeutic module; a vibration generator; a control panel; an ozone module; and interchangeable spreading cap configured to hold and spread a gel, an ointment, a cream or a viscous liquid comprising one or more of the foregoing, and wherein the negative ion module, phototherapeutic module, the vibration generator and the ozone generator in electronic communication with the power supply and the control panel, and further wherein the device, using the control panel, is configured to selectively release negative ions through the slits disposed in the housing cap and/or the at least one air outlet slot in the ventral housing portion, and/or vibrate the housing cap, and/or emit phototherapeutic electromagnetic radiation through the lens; contacting the skin portion where permeation of a cream, an ointment, a gel, or a viscous liquid is sought to be improved; and using the control panel, selectively releasing negative ions through the slits disposed in the housing cap or the at least one air outlet slot in the ventral housing portion, vibrating the housing cap, emitting phototherapeutic electromagnetic radiation through the lens, generating ozone; and using the spreading cap, spreading the gel, ointment, cream or the viscous liquid comprising one or more of the foregoing. The spreading cap can be transparent and/or disposable and be made of an appropriate material such as a clear (potentially reinforced) thermoplastic material or, for example, glass.

These and other features of the a portable device capable of simultaneously and selectively generate a plurality of treatment modes for headaches and mood disorders at varying intensities as described herein, will become apparent from the following detailed description when read in conjunction with the drawings, which are exemplary, not limiting, and wherein like elements are numbered alike in several figures.

### BRIEF DESCRIPTION OF THE DRAWINGS

For a better understanding of the portable device capable of simultaneously and selectively generate a plurality of treatment modes for headaches and mood disorders described herein, with regard to the embodiments thereof, reference is made to the accompanying drawings, in which like numerals designate corresponding elements or sections throughout and in which:

FIG. 1, is a bottom right isometric view of an embodiment of the portable device described herein;

FIG. 2, is an illustration of an X-Y cross section of FIG. 1;

FIG. 3, is an illustration of a side view of FIG. 1;

FIG. 4, is an illustration of a Y-Z cross section of an embodiment of the portable device of FIG. 3;

FIG. 5, is an illustration of a X-Z cross section of an embodiment of the portable device of FIG. 4, along line C-C in FIG. 3 (5A), and along line D-D in FIG. 3 (5B);

FIG. 6, is an illustration of an embodiment of the portable device, further comprising a massaging housing cap (FIG. 6A), a spreading cap configured to further allow better penetration of light into the skin, applying a topical cream, ointment or gel with or without medication (FIG. 6B), and integral housing cap (FIG. 6C);

FIG. 7, is an illustration of an embodiment of the devices action in treating headaches and mood disorders; and

FIG. 8 is an illustration of an embodiment of the portable device's action on a portion of the skin to improve penetration of cream, ointment, or gel with or without medication.

### DETAILED DESCRIPTION AS DISCLOSED AND CLAIMED HEREIN

Provided herein are embodiments of device for the non-invasive treatment (encompassing also prophylaxis, therapy and cure) of, for example, headaches and mood disorders. In addition, the device provided herein may have a synergistic effect with various medicines, such that its use in combination with other medication may reduce the amount of medicine needed to obtain the same therapeutic effect, thus reduce cost of treatment and reduce adverse side effects. More specifically, provided herein are embodiments of portable device capable of simultaneously and selectively generate at least three modes of treatment of headaches and mood disorders at varying intensities. For example, the portable device provided herein can have a combination of the following functions: vibration, emission of red light, targeted release and regulation of negative ions and ozone atmosphere around its most effective area. Typically, when available, the negative ions and ozone are released without individual control. The device described herein makes it possible to control the distance the charged air and ozone travel to be inhaled. This can be achieved by using have the fan where the user can control, adjust, and regulate speed, which will allow regulation of the air with the proper concentration of negative ions and or ozone to reach a distance of, for example, between about 2.0 cm and about 25 cm. Moreover, the presence of ozone at the concentrations provided can sterilize the air without having toxic effects on the user.

The portable device described herein can have a substantially elongated housing, the housing comprising: a dorsal housing portion (in other words referring to the back of the portable device) and a ventral (in other words, the portion opposite the dorsal portion) housing portion, each housing portion having a proximal end and a distal end. When coupled, the dorsal and ventral housing portions define a cylindrical structure having a longitudinal axis with a length L that is larger than the diameter D (i.e., L/D>1). An air inlet (in other words, an air intake cavity) opening can be disposed in each of the dorsal housing portion and the ventral housing portion. The air intake opening can have a generally semi-circular, or oval cross section, and be comprised of an indentation in the dorsal and ventral housing portions. The opening can have at least one air outlet (in other words, an exhaust) slot disposed in the ventral housing portion and located distal to the ventral air inlet opening between the ventral intake opening and the distal end of the ventral portion. Although the shapes of the various slots and openings are described, other shapes for each opening described herein are also contemplated and the shape provided should not be limiting.

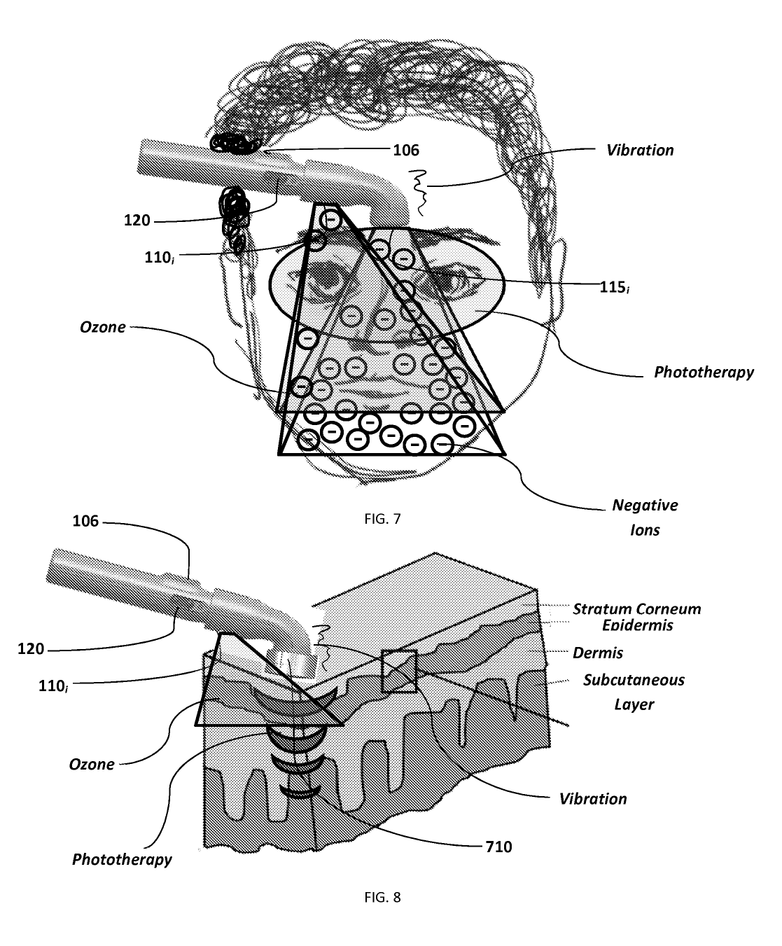

The portable device further comprises a housing cap, configured to be operably coupled to the distal end of each of the dorsal housing portion and the ventral housing portion. The housing cap can have an annular structure with a lip configured to receive a shelf defined in the distal end of both the dorsal and ventral housing portions in the proximal end of the housing cap, the distal end of the housing cap can comprise a translucent or transparent, optionally removable lens and can define a plurality of arcuate slits in the periphery of the lens. Again, while the slits are described as arcuate, other slit shapes (straight, angular, holes, etc., are also contemplated). In an embodiment, the lens can be coupled onto a bracket configured to receive the lens, wherein the bracket protrude from the housing cap, so when it abuts the forehead or any other organ, air with the negative ions and/or ozone will exist through the slits and combine with the outlet slots disposed in the ventral housing portion and create a zone/area (see e.g., FIG. 7) of purified and sterile air that can be controlled by speed of fan (to about 2.0 cm and about 25 cm) around the head or organ, thus providing a controlled atmosphere around, for example, the eyes and nose. Further control of the fan speed can be monitored using a digital display coupled centrally on the dorsal housing portion. Other adaptor caps can be caps for massaging and for spreading creams and/or ointments and/or gels, with or without medication. Both adaptor caps (e.g., the spreading cap), can be translucent and/or disposable.

Unlike devices currently existing, there is no waste of negative ions and/or ozone to locations where their efficacy is negligible. Moreover, changing the position of the device will allow the negative ions and/or the ozone to be expelled from the opening at the ventral housing portion.

The term “translucent” indicates in an embodiment, that light can pass through the lens, but the light is diffused. It does not require that a whole surface of the lens itself is transparent and portions of the lens may be transparent or opaque, for example to serve a function or to form a decorative pattern. The term “translucent” as used herein would refer to a composition that transmits at least 60% in the region ranging from 600 nm to 900 nm with a haze of less than 40%. In one embodiment, the composition has a transmission of at least 75%. In another embodiment, the composition has a transmission of at least 85%. In yet another embodiment, the composition has a haze of less than 40%, and in another embodiment, the composition has a haze of less than 10%. In another embodiment, the composition has a haze of less than 5%.

The term “transparent” refers to a lens composition capable of at least 70% transmission of light. The light referred to can be, e.g., emitted light (e.g., from a fluorochrome), with transmittance of at least 80%, for example at least 85%, or at least 90%, as measured spectrophotometrically using water as a standard (100% transmittance) at 690 nm. The term “transparent” as used herein would also refer to a lens composition that transmits at least 70% in the region ranging from about 600 nm to about 900 nm with a haze of less than 10%.

The term “haze” as used herein refers to the percentage of diffused light transmitted by a material (e.g., the lens) measured according to the ASTM D 1003 standard. In an embodiment, the term “haze” refers to that percentage of light which in passing through deviates from the incident beam greater than 2.5 degrees on the average. “Haze” may be measured herein by a Byk Gardner haze meter (all haze values herein are measured by such a haze meter and are given as a percentage of light scattered).

As indicted, the lens present in the housing cap, can be a removable and be configured to filter visible light spectrum, providing incident light at a predetermined wavelength, for example, between about 600 nm and about 900 nm. The term “filter” refers to a material (e.g., the lens composition) that prevents the transmission of some wavelengths of visible light through the filter material, regardless of the nature of the prevention mechanism. Thus, a filter herein can block some wavelengths, absorb some wavelengths, scatter some wavelengths, or otherwise interact with incident light to prevent transmission through the filter material of some of the wavelengths included in the incident radiation or light.

The dorsal housing portion can be operably coupled to the ventral housing portion so as to define at least one compartment between the dorsal housing portion and the ventral housing portion. Complimentary partitioning walls defined in the dorsal and ventral housing portions can be configured to define discrete compartments within the housing once the dorsal and ventral housing portions are coupled, such that the compartments are in communication with each other. Optionally, the dorsal and ventral housing portions can have an opening defined in the proximal end of the ventral housing portion; a power supply. Moreover, the dorsal housing portion can comprise and have coupled to, a digital display, for example LCD (liquid crystal display), in communication with the control unit and the device processor (or CPU), configured to display information relating to, for example, fan speed, negative ion concentration, ozone concentration, phototherapeutic module's emission wavelength or a combination of displays comprising one or more of the foregoing. Accordingly and in an embodiment, the device further comprises a processor having stored thereon executable commands configured to operate the device provided herein to perform the functions described herein.

The portable device capable of simultaneously and selectively generate at least three modes of treatment can have a negative ion module. The negative ion module can be for example, a corona discharge type ion generator, or an electron emission type ion generator (in other words, where pulse type high voltage is applied to a discharge unit to directly emit electrons in air, thereby generating negative ions), an ion generator using a Lenard effect, or an ion generator using a-rays in other examples of the negative ion module described herein. The negative ion module can have a driver (for example, a motor operably coupled to a gear assembly) with a drive body and a drive shaft, in electronic communication with the power supply and the control panel. The drive shaft can be operably coupled to the fan, which can be disposed distally to the air inlet openings defined in each of the dorsal housing portion and ventral housing portion. The negative ion module can further have a negative ion generator comprising a drive circuit in electronic communication with the power supply; a high voltage transformer operably coupled to the drive circuit, wherein the high voltage transformer is configured to amplify electromagnetic coupling via electromagnetic induction; a rectifying circuit operably coupled to the high voltage transformer, configured to rectify the transformer output into a DC voltage; and a plurality of electric discharge electrodes disposed proximally to the fan. The negative ion generator (for example, an electron emission, or corona discharge ion generator) can be configured to generate negative ions at a density of between about 1×10⁴ negative ions/cm³ and about 3×10⁶ negative ions/cm³ at a distance of between about 2.0 cm and about 25 cm from the slits in the housing cap.

The portable device capable of simultaneously and selectively generate at least three modes of treatment can have a phototherapeutic module, which can comprises a light emitting diode (LED) operably coupled on a carrying frame, configured to emit electromagnetic radiation through the lens at a selectable, predetermined wavelength and intensity, the carrying frame operably coupled to the housing cap. The wavelength can be the whole visible spectrum (e.g., white LED, or white organic light emitting diode, WOLED) with a filter lens that will allow a predetermined wavelength to be emitted from the lens. The lens can also be transparent lens, and the LED can emit light at a wavelength of between about 600 nm and about 650 nm, or between about 615 nm and about 645 nm, for example, between about 620 nm and about 640 nm, or between about 630 nm and about 640 nm. In an embodiment, the selectable predetermined wavelength can be between about 600 nm and about 900 nm with an intensity of about 2.5 and about 10 Candela at a distance of between about 2.5 cm and about 25 cm from the lens. Moreover, the light emitting diode (LED, e.g., OLED) can be coupled to a potentiometer and be configured to selectively operate as a strobe (in other words, provide flashes of high intensity light emission for a short duration) having a variable strobe rate of between 1.0 and 60 Hz. The strobe length can be exhibited on the display. The lens can be coupled to a bracket configured to receive the lens. The bracket can protrude from the external surface of the housing cap, such that when in use, opening, e.g., arcuate slits defined around the lens are NOT blocked.

Likewise, the portable device capable of simultaneously and selectively generate at least three modes of treatment can have a vibration generator, which can comprise a weighted cylinder selectively couplable (in other words, based on the users choice the selectively coupleable, or “selectively coupled”, can be activated or deactivated as desired without affecting other components, elements or modules) to the drive shaft of the motor, the weighted cylinder being coupleable to the drive shaft between the fan and the motor body. Upon activation, the vibrator can produce vibration at a tunable frequency of between about 800 Hz and about 3000 Hz (3.0 kHz). Likewise, the vibration stroke can be between about 0.5 mm and about 2.0 mm. Typically, while vibration frequency can be tunable (in other words, selectable), stroke length will be fixed, for example 1.0 mm.

Moreover, the portable device capable of simultaneously and selectively generate at least three modes of treatment can also have a control panel and optionally an ozone (i.e., the triatomic form of oxygen) module. The ozone module, when present, for example, produces an AC corona discharge in a gap bounded by metallic electrodes (e.g., metal brush) and containing at least one solid dielectric barrier. The gap can be between about 1 and 5 mm and the intake air can be passed longitudinally along the gap. The ozone generator can be configured to generate between about 11 (e.g., 0.23 mol./cm³) g/cm³ and about 70 (e.g., 1.46 mol./cm³) g/cm³ (units interchangeable with g/ml) of ozone at a distance of between about 2.0 cm and about 25 cm from the slits in the housing cap. At these concentrations, the Ozone is configured to sterilize the air around the mouth and nose, without rising to known toxic levels if inhaled. When used for example in the treatment of wounds, the ozone generator can be configured to release a higher ozone concentration, for example between about 100 g/cm³ and about 150 g/cm³, for example, 125 g/cm³ directly in the vicinity of the open wound thus sterilizing the treatment area.

The negative ion module, phototherapeutic module, the vibration generator and the ozone generator can be each compartmentalized in the compartments defined in by the various partitioning walls defined in the dorsal and ventral housing portion and be in electronic communication with both the power supply (e.g., a battery, or a rechargeable battery); and the control panel and alternatively or in addition, to a central processing unit (CPU). Likewise, the portable device capable of simultaneously and selectively generate at least three modes of treatment can have a selectable AC-DC converter configured to provide DC voltage of between 1.0 and 10 Volts, the converter being in communication with a transformer port defined in the proximal end of the ventral housing portion. The device can also have an adapter, configured to couple the device to the head of the subject at the proper gap, configured to optimize synergistic effects of the various treatment modes.

The device, using the control panel, is configured to selectively (in other words, any permutation of treatment modes can be activated/deactivated as desired); release negative ions through the slits disposed in the housing cap and/or the at least one air outlet slot in the ventral housing portion, and/or vibrate the housing cap, and/or emit phototherapeutic electromagnetic radiation through the transparent, or translucent lens, as well as optionally generate ozone at safe concentration at a distance can be predetermined using the adapter described herein (e.g., between 1.0 cm and about 25 cm, or between about 5.0 cm and about 15 cm. In addition, the housing cap can be coupled to a spreading cap used to improve penetration of creams, ointments, and gels with or without medication into the skin. The spreading cap can be made of a transparent material. The purpose for the transparency is twofold. Initially, the transparency allows the light generated (e.g., red light) to pass through, and allow better penetration of light into the skin (since the light is being emitted directly at the point of contact with the skin). Also, this will allow illumination of the surface so the user can see (more clearly) whether there is any cream left. This spreading cap (in other words, the spreading cap can also be disposable or be selectively removable and interchangeable by each individual if few people use the same device).

The combined and synergistic action of the device can facilitate the penetration of the creams and/or ointments and/or gels, with or without medication to e depth of between about 3 mm to about 20 mm. This can be achieved, by using the vibrator which locally stretches the skin, increasing surface area for penetration, while simultaneously controlling the sterility of the ointment/cream by streaming ozone to the affected area, thus assisting in nullifying any contamination that may have occurred due to the removal of the ointment/cream/gel from its container. (See e.g., FIG. 8) The cream/ointment/gel, can be administered initially without the portable device described herein while still being affected by its use on the affected area. For example, creams used for the prevention of scar formation post-op.

In an embodiment, the portable device capable of simultaneously and selectively generate at least three modes of treatment can be used in the methods described herein. Accordingly and in an embodiment, provided herein is a method of treating a mood disorder (e.g., seasonal affective disorder (SAD), mild depression, insomnia and the like), a headache or their combination in a subject in need thereof, comprising: providing a device comprising; a substantially elongated housing, the housing comprising: a dorsal housing portion and a ventral housing portion, each housing portion having a proximal end and a distal end; an air inlet opening disposed in each of the dorsal housing portion and the ventral housing portion; at least one air outlet slot disposed in the ventral housing portion and located between the ventral air inlet opening and the distal end of the ventral portion; a housing cap, operably coupled to the distal end of each of the dorsal housing portion and the ventral housing portion, the housing cap comprising a lens and defines a plurality of arcuate slits in the periphery of the lens, wherein the dorsal housing portion is operably coupled to the ventral housing portion so as to define at least one compartment between the dorsal housing portion and the ventral housing portion; and a display panel coupled to the dorsal housing portion; a power supply; a fan; a negative ion module; a phototherapeutic module; a vibration generator; a control panel; and optionally an ozone module, wherein the negative ion module, phototherapeutic module, the vibration generator and optionally the ozone generator can each be in electronic communication with the power supply and the control panel (and/or the CPU), wherein the device, using the control panel, is configured to selectively release negative ions through the slits disposed in the housing cap and/or the at least one air outlet slot in the ventral housing portion, and/or vibrate the housing cap, and/or emit phototherapeutic electromagnetic radiation through the lens; placing the distal end of the device abutting the forehead of the subject; and using the control panel, selectively releasing negative ions through the slits disposed in the housing cap or the at least one air outlet slot in the ventral housing portion, vibrating the housing cap, emitting phototherapeutic electromagnetic radiation through the lens, optionally generating ozone, or effecting any combination of at least four of the foregoing. For example, negative ions at 1×10⁵/cm³+light emission strobe at 635 nm and 5 Candela+vibration for treatment of seasonal affective disorder, or in another example, releasing negative ions at 2.5×10⁶/cm³+light emission, 30 Hz strobe at 680 nm and 10 Candela+generating ozone at 25 g/cm3 for the treatment of headache.

The term “disorder” in the sense of mood disorders, refers to anomalies that are generally seen as preliminary disease states and which manifest themselves in the form of certain signs, symptoms and/or dysfunctions. The treatment can be directed to single disorders, in other words, anomalies or disease states, but multiple anomalies, which can possibly be causally connected to one another, can also occur together as patterns, in other words, syndromes, which can be treated according to the provided methods. This state can be temporary, progressive or permanent. The subject receiving this treatment is any animal in need, including primates, in particular humans, and other mammals such as equines, cattle, swine and sheep; and poultry and pets in general.

Also, provided herein is a method of increasing the permeability of at least a portion of the skin, comprising: providing a device comprising; a substantially elongated housing, the housing comprising: a dorsal housing portion and a ventral housing portion, each housing portion having a proximal end and a distal end; an air inlet opening disposed in each of the dorsal housing portion and the ventral housing portion; at least one air outlet slot disposed in the ventral housing portion and located between the ventral air inlet opening and the distal end of the ventral portion; a housing cap, operably coupled to the distal end of each of the dorsal housing portion and the ventral housing portion, the housing cap comprising an interchangeable lens and a plurality of arcuate slits defined in the periphery of the lens; a spreading cap, operably coupled to the housing cap and configured to hold and spread a gel, an ointment, a cream or a viscous liquid comprising one or more of the foregoing, wherein the dorsal housing portion is operably coupled to the ventral housing portion so as to define at least one compartment between the dorsal housing portion and the ventral housing portion; a power supply; and a display panel coupled to the dorsal housing portion; a fan; a negative ion module; a phototherapeutic module; a vibration generator; a control panel; and an ozone module, wherein the negative ion module, phototherapeutic module, the vibration generator and the ozone generator in electronic communication with the power supply and the control panel, wherein the device, using the control panel, is configured to selectively release negative ions through the at least one air outlet slot in the ventral housing portion, and/or vibrate the housing cap, and/or emit phototherapeutic electromagnetic radiation through the lens; contacting the skin portion where permeation of a cream, an ointment, a gel, or a viscous liquid is sought to be improved; and using the control panel, selectively releasing negative ions through the slits disposed in the housing cap or the at least one air outlet slot in the ventral housing portion, vibrating the housing cap, emitting phototherapeutic electromagnetic radiation through the lens, generating ozone; and using the spreading cap, spreading the gel, ointment, cream or the viscous liquid comprising one or more of the foregoing.

A more complete understanding of the components, methods, and devices disclosed herein can be obtained by reference to the accompanying drawings. These figures (also referred to herein as “FIG.”) are merely schematic representations based on convenience and the ease of demonstrating the present disclosure, and are, therefore, not intended to indicate relative size and dimensions of the devices or components thereof, their relative size relationship and/or to define or limit the scope of the exemplary embodiments. Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function. Likewise, cross sections are referred to on normal orthogonal coordinate system having XYZ axis, such that Y axis refers to front-to-back, X axis refers to side-to-side, and Z axis refers to up-and-down.

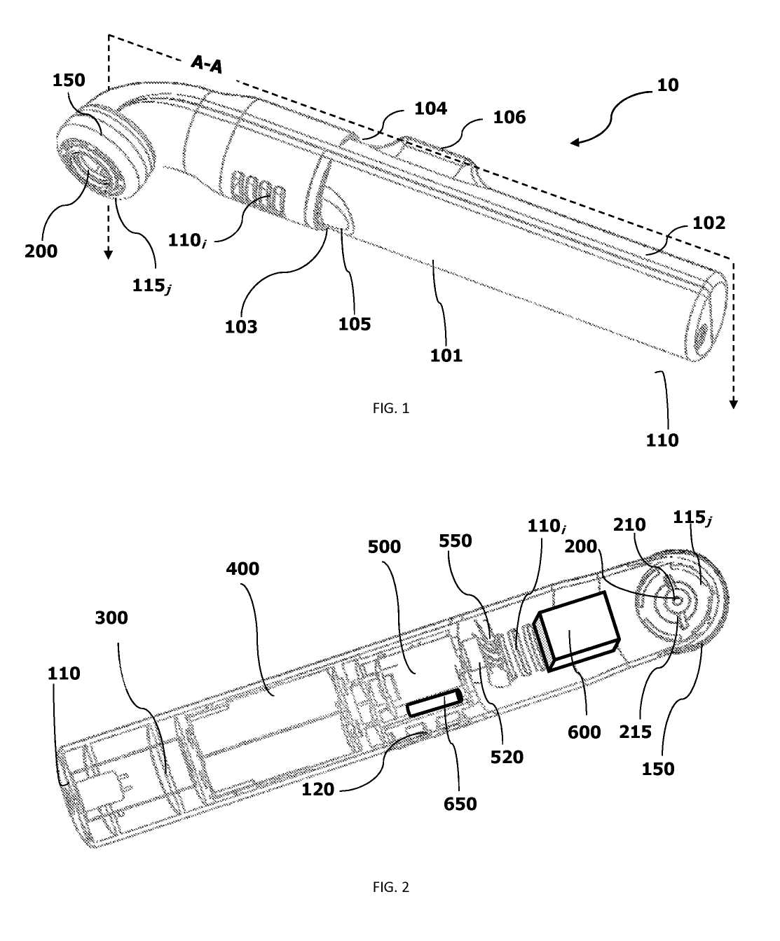

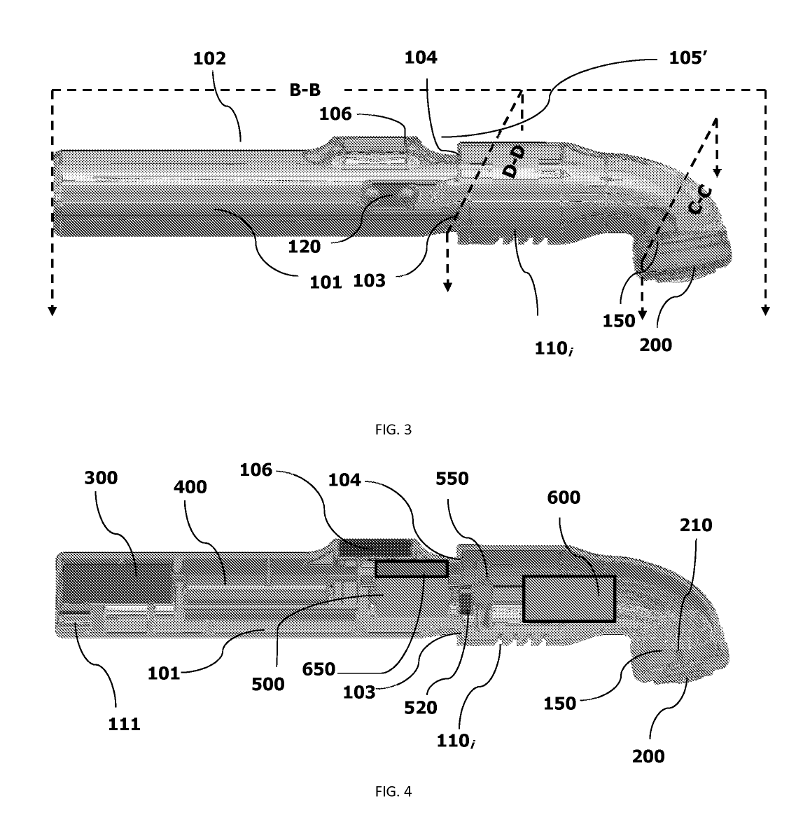

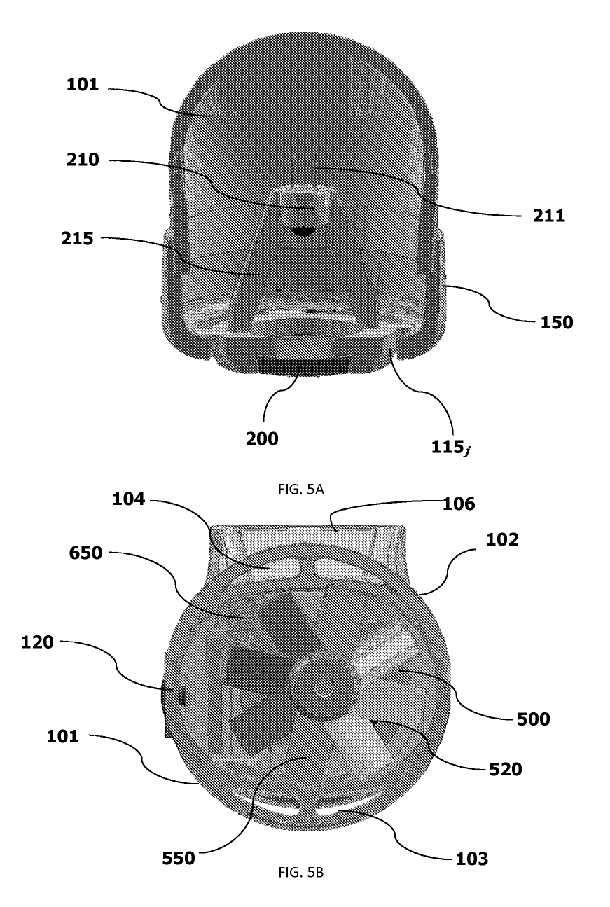

Turning now to FIGS. 1-8, illustrating in FIG. 1, a bottom right perspective view of portable device 10, having a substantially elongated housing (FIGS. 1, 3), the housing comprising: dorsal housing portion 102 and ventral housing portion 101, each housing portion having a proximal end and a distal end; air inlet opening 104, 103 disposed in each of dorsal housing portion 102 and ventral housing portion 101 (FIGS. 4, 5B). At least one air outlet slot 110i (FIG. 3) is disposed in ventral housing portion 101 and located between ventral air inlet opening 103, and the distal end of the ventral housing portion 101. As illustrated (e.g., FIG. 1) housing cap 150, can be operably coupled to the distal end of each of dorsal housing portion 102 and ventral housing portion 101, interchangeable housing cap 150 comprising lens 200 (see e.g., FIGS. 1, 5A, 6C) and defines plurality of arcuate slits 115j in the periphery of lens 200. As illustrated in, for example FIG. 4, dorsal housing portion 102 can be operably coupled to ventral housing portion 101 so as to define at least one compartment between dorsal housing portion 102 and ventral housing portion 101; and optionally opening 110 defined in the proximal end of ventral housing portion 101. Moreover, dorsal housing portion can further comprise a digital display 106 (FIGS. 1, 3, 4, 5B, 7, 8) operably coupled to dorsal housing portion 102. As illustrated, the device comprises power supply 400 (FIGS. 2, 4); negative ion module 300; phototherapeutic module 210; vibration generator 520; control panel 120 (FIGS. 2, 3); and optionally ozone module 600, 650, FIGS. 2, 4), wherein negative ion module 300, phototherapeutic module 200,210, vibration generator 520 and optionally ozone generator 600, 650 being in electronic communication with both power supply 400 and control panel 120. The device, by effecting the control panel, can be configured to selectively release negative ions through slits 115ⱼ (FIG. 1) disposed in housing cap 150 and/or at least one air outlet slot 110ᵢ in ventral housing portion 101, and/or vibrate housing cap 150, and/or emit phototherapeutic electromagnetic radiation through lens 200.

As illustrated in FIG. 5A, the phototherapeutic module can comprise light emitting diode (LED) 210 having leads 211 all on illuminating housing cap 150 configured to couple to both power source 400 and control panel 120, being operably coupled on carrying frame 215, configured to emit electromagnetic radiation through lens 200 at a selectable, predetermined wavelength, duration and intensity, carrying frame 215 operably coupled to illuminating housing cap 150.

As illustrated further, wherein the negative ion module can comprise driver 500 with a drive body and a drive shaft, in electronic communication with both power supply 400 and control panel 120 (FIGS. 1, 2, 5A). Fan 550 can be operably coupled to driver's 500 drive shaft, fan 550 being disposed distally to air inlet openings 104, 103 defined respectively in dorsal housing portion 102 and ventral housing portion 101; and negative ion generator 300 comprising a drive circuit in electronic communication with power supply 400; a high voltage transformer operably coupled to the drive circuit, wherein the high voltage transformer is configured to amplify electromagnetic coupling via electromagnetic induction; a rectifying circuit operably coupled to the high voltage transformer, configured to rectify the transformer output into a DC voltage; and plurality of electric discharge electrodes 650 disposed proximally to fan 550.

As further illustrated in FIG. 4, the vibration generator comprises a weighted cylinder 520 selectively couplable to the drive shaft of driver 500, weighted cylinder 520 being coupleable to the drive shaft between fan 550 and driver 500 body. Moreover, illustrated is a selectable AC-DC converter 111 configured to provide DC voltage of between 1.0 and 10 Volts, converter 111 being in communication with a transformer port opening 110.

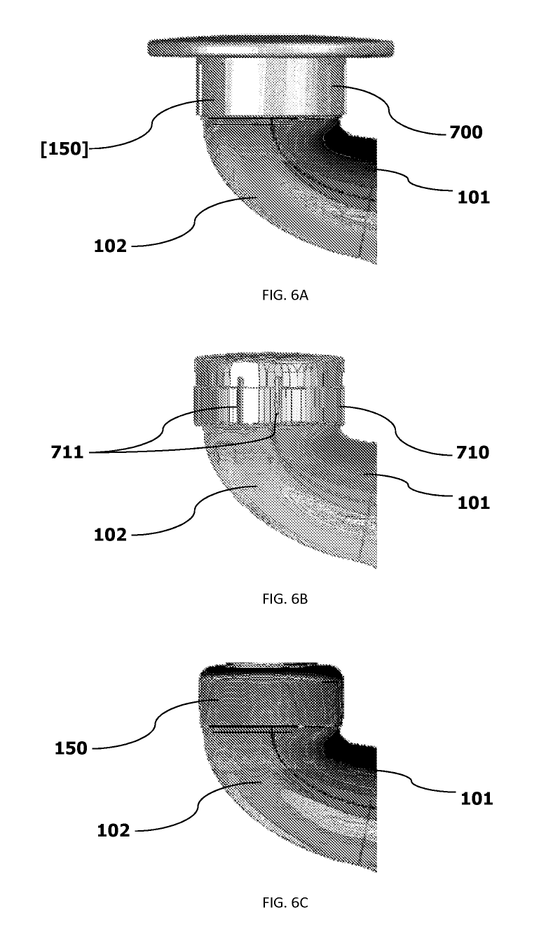

Turning now to FIG. 6, illustrating in FIG. 6A, device 10 further comprising removable interchangeable massaging cap 700, the massaging cap being operably coupled to housing cap 150 and can be configured to abut the head of the subject (see e.g., FIG. 7). FIG. 6B illustrates spreading cap 710 having spreading slits 711, configured to hold and spread cream, gel, or ointment and other viscous liquids (e.g., apparent viscosity of between about 1.5×10¹ cps, to about 5.0×10⁵ cps, depending on vibration (fan) speed) in a sterile environment and be disposable and clear/transparent. Spreading cap 710 is configured to operably couple to housing cap 150, allowing ozone and negative ions to be released from outlet slots 110ᵢ. (See e.g., FIG. 8)

The device can further comprise a central processing unit operably coupled to control panel 120 and display panel 106, having a processor, a microprocessor, a controller, a microcontroller, a chip, or other processing device that carries out arithmetic and logic instructions of an operating system, a computer program, an application, or the like. According to various embodiments disclosed herein, the CPU can comprise a processing unit (typically including an arithmetic logic unit and a control unit) and a memory (also known as “registers,” or Read Only Memory (ROM)). The CPU may also comprise a memory storing an operating system and/or any further executable instructions and/or data configured to affect the function of the components described herein, according to the methods provided. The memory can be implemented within the CPU or externally. For example, the CPU may share a single memory coupled to a bus. As used herein, the term “memory” refers to any type of long term, short term, volatile, nonvolatile, or other storage devices and is not limited to any particular type of memory or number of memories, or type of media upon which memory is stored. In an embodiment, the CPU can be configured to execute computer-executable instructions, effecting the device to selectively release negative ions through the slits disposed in the housing cap and/or the at least one air outlet slot in the ventral housing portion, and/or vibrate the housing cap, and/or emit phototherapeutic electromagnetic radiation through the lens.

Turning now to FIG. 7, illustrating an embodiment of the methods described herein where portable device 10 is placed abutting the forehead of the subject and using control panel 120, selectively emitting electromagnetic radiation around the eyes, either steady or as strobe light, while simultaneously releasing negative ions, sterilizing the air with ozone and vibrating the housing cap at, for example, 1 kHz. All functions can be monitored and regulated using display panel 106.

Turning now to FIG. 8, illustrating an embodiment of the method for increasing the permeability of at least a portion of the skin, where portable device 10 is equipped with transparent and/or disposable spreading cap 710 operably coupled to housing cap 150 (not shown, see e.g., FIG. 6C), and while vibrating transparent spreading cap 710, thereby stretching the skin at the point of contact, simultaneously illuminating the location with electromagnetic radiation configured to increase permeability of the cream/ointment/gel as it is spread from slits 711 (see e.g., FIG. 6B) onto the skin and while ozone is released from outlet slots 110ᵢ to sterilize the area of application of the viscous fluid.

The term “about”, when used in the description of the technology and/or claims means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such and may include the end points of any range provided including, for example ±25%, or ±20%, specifically, ±15%, or ±10%, more specifically, ±5% of the indicated value of the disclosed amounts, sizes, formulations, parameters, and other quantities and characteristics.

All ranges disclosed herein are inclusive of the endpoints, and the endpoints are independently combinable with each other. “Combination” is inclusive of blends, mixtures, alloys, reaction products, and the like. Furthermore, the terms “first,” “second,” and the like, herein do not denote any order, quantity, or importance, but rather are used to denote one element from another. The terms “a”, “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the slit(s) includes one or more slit). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The term “module” may refer to at least a self-contained component (unit, system or element) that is used in combination with other components and/or a separate and distinct unit of hardware and/or software that may be used as a component in a device, such as a portable device capable of simultaneously and selectively generate at least three modes of treatment of headaches and mood disorders at varying intensities. The term “module” may also refer to at least a self-contained assembly of electronic components and circuitry, such as a circuit configured to control the wavelength, intensity and duration of an LED light that is installed as a unit. The term “module” may be used interchangeably with the term “unit.” In an embodiment, the term “module” is separate and contain the term “generator”

Accordingly, provided herein is a portable device for treating a mood disorder, a headache or their combination in a subject in need thereof, the device comprising: a substantially elongated housing, the housing comprising: a dorsal housing portion and a ventral housing portion, each housing portion having a proximal end and a distal end; an air inlet opening disposed in each of the dorsal housing portion and the ventral housing portion; at least one air outlet slot disposed in the ventral housing portion and located between the ventral air inlet opening and the distal end of the ventral portion; a housing cap, operably coupled to the distal end of each of the dorsal housing portion and the ventral housing portion, the housing cap comprising an interchangeable lens and defines a plurality of arcuate slits in the periphery of the lens, wherein the dorsal housing portion is operably coupled to the ventral housing portion so as to define at least one compartment between the dorsal housing portion and the ventral housing portion; and a display panel coupled to the dorsal housing portion; a power supply; a fan; a negative ion generating module; a phototherapeutic module; a vibration generator; a control panel; and optionally an ozone module, wherein the negative ion module, phototherapeutic module, the vibration generator and optionally the ozone generator are each in electronic communication with the power supply and the control panel, and wherein the device, using the control panel, is configured to selectively release negative ions through the slits disposed in the housing cap and/or the at least one air outlet slot in the ventral housing portion, and/or vibrate the housing cap, and/or emit phototherapeutic electromagnetic radiation through the lens, wherein (i) the phototherapeutic module comprises a light emitting diode (LED) operably coupled on a carrying frame, configured to emit electromagnetic radiation through the lens at a selectable, predetermined wavelength, duration and intensity, the carrying frame operably coupled to the housing cap, (ii) the selectable predetermined wavelength is between about 600 nm and about 900 nm with an intensity of about 2.5 and about 10 Candela at a distance of between about 2.5 and about 25 cm from the lens, (iii) the light emitting diode is configured to selectively operate as a strobe having a variable strobe rate of between 1.0 and 60 Hz, wherein (iv) the negative ion module comprises: a driver with a driver body and a drive shaft, in electronic communication with the power supply and the control panel; a fan operably coupled to the motor's drive shaft, the fan disposed distally to the air inlet openings defined in each of the dorsal housing portion and ventral housing portion; and a negative ion generator comprising a drive circuit in electronic communication with the power supply; a high voltage transformer operably coupled to the drive circuit, wherein the high voltage transformer is configured to amplify electromagnetic coupling via electromagnetic induction; a rectifying circuit operably coupled to the high voltage transformer, configured to rectify the transformer output into a DC voltage; and a plurality of electric discharge electrodes disposed proximally to the fan, (v) the negative ion generator is configured to generate negative ions at a density of between about 1×10⁵ negative ions/cm³ and about 3×10⁶ negative ions/cm³ at a distance of between about 2.0 cm and about 25 cm from the slits in the housing cap, wherein (vi) the vibration generator comprises a weighted cylinder selectively couplable to the drive shaft of the motor, the weighted cylinder being coupleable to the drive shaft between the fan and the motor body, configured to vibrate at a frequency of between about 800 Hz, and about 3000 Hz, wherein (vii) the ozone module, is configured to generate between about 11 and about 70 g/cm3 of ozone at a distance of between about 2.0 cm and about 25 cm from the slits in the housing cap, wherein (viii) the housing cap configured to operably couple to the dorsal and ventral housing portion is configured to provide the device with an additional functional module, (ix) for example a spreading cap operably coupled to the housing cap, comprises a receptacle; and slits configured to provide a spreading functionality to a cream, ointment, gel or a viscous liquid comprising one or more of the foregoing, wherein (x) the housing cap comprises a filter configured to modulate the wavelength emitted by the phototherapeutic module and increase skin permeability, and wherein (xi) the device further comprising a selectable AC-DC converter configured to provide DC voltage of between 1.0 and 10 Volts, the converter being in communication with a transformer port defined in the proximal end of the ventral housing portion.

In another embodiment, provided herein is a method of treating a mood disorder, a headache or their combination in a subject in need thereof, comprising: providing a device comprising; a substantially elongated housing, the housing comprising: a dorsal housing portion and a ventral housing portion, each housing portion having a proximal end and a distal end; an air inlet opening disposed in each of the dorsal housing portion and the ventral housing portion; at least one air outlet slot disposed in the ventral housing portion and located between the ventral air inlet opening and the distal end of the ventral portion; a housing cap, operably coupled to the distal end of each of the dorsal housing portion and the ventral housing portion, the housing cap comprising a lens and defines a plurality of arcuate slits in the periphery of the lens, wherein the dorsal housing portion is operably coupled to the ventral housing portion so as to define at least one compartment between the dorsal housing portion and the ventral housing portion; and a display panel coupled to the dorsal housing portion; a power supply; a fan; a negative ion module; a phototherapeutic module; a vibration generator; a control panel; and an ozone module, wherein the negative ion module, phototherapeutic module, the vibration generator and the ozone generator are each in electronic communication with the power supply the display, and the control panel, wherein the device, using the control panel, is configured to selectively release negative ions through the slits disposed in the housing cap and/or the at least one air outlet slot in the ventral housing portion, and/or vibrate the housing cap, and/or emit phototherapeutic electromagnetic radiation through the lens; placing the distal end of the device abutting the forehead of the subject; and using the control panel, selectively releasing negative ions through the slits disposed in the housing cap or the at least one air outlet slot in the ventral housing portion, vibrating the housing cap, emitting phototherapeutic electromagnetic radiation through the lens, optionally generating ozone, or effecting any combination of at least three of the foregoing., wherein (xii) the phototherapeutic module comprises a light emitting diode (LED) operably coupled on a carrying frame, configured to emit electromagnetic radiation through the lens at a selectable, frequency of between about 600 nm and about 900 nm with an intensity of about 2.5 and about 10 Candela at a distance of between about 2.5 and 25 cm from the lens, the carrying frame operably coupled to the housing cap and wherein the light emitting diode is configured to selectively operate as a strobe having a variable strobe rate of between 1.0 and 60 Hz, (xiii) the negative ion generator is configured to generate negative ions at a density of between about 1×10⁵ negative ions/cm³ and about 3×10⁶ negative ions/cm³ at a distance of between about 2.0 cm and about 25 cm from the slits in the housing cap, wherein and wherein (xiv) the ozone module, is configured to generate between about 11 and about 70 g/cm3 of ozone at a distance of between about 2.0 cm and about 25 cm from the slits in the housing cap.

In yet another embodiment, provided herein is a method of increasing the permeability of at least a portion of the skin, comprising: providing a device comprising; a substantially elongated housing, the housing comprising: a dorsal housing portion and a ventral housing portion, each housing portion having a proximal end and a distal end; an air inlet opening disposed in each of the dorsal housing portion and the ventral housing portion; at least one air outlet slot disposed in the ventral housing portion and located between the ventral air inlet opening and the distal end of the ventral portion; a housing cap, operably coupled to the distal end of each of the dorsal housing portion and the ventral housing portion, the housing cap comprising an interchangeable lens and a plurality of arcuate slits defined in the periphery of the lens; a spreading cap operably coupled to the housing cap, configured to hold and spread a gel, an ointment, a cream or a viscous liquid comprising one or more of the foregoing, wherein the dorsal housing portion is operably coupled to the ventral housing portion so as to define at least one compartment between the dorsal housing portion and the ventral housing portion; a power supply; and a display panel coupled to the dorsal housing portion; a negative ion module; a phototherapeutic module; a vibration generator; a control panel; and an ozone module, wherein the negative ion module, phototherapeutic module, the vibration generator and the ozone generator are each in electronic communication with the power supply, the display panel, and the control panel, wherein the device, using the control panel, is configured to selectively release negative ions through the slits disposed in the housing cap and/or the at least one air outlet slot in the ventral housing portion, and/or vibrate the housing cap, and/or emit phototherapeutic electromagnetic radiation through the lens; contacting the skin portion where permeation of a cream, an ointment, a gel, or a viscous liquid is sought to be improved; and using the control panel, selectively releasing negative ions through the slits disposed in the spreading cap or the at least one air outlet slot in the ventral housing portion, vibrating the housing cap, emitting phototherapeutic electromagnetic radiation through the lens, generating ozone; and using the spreading cap, spreading the gel, ointment, cream or the viscous liquid comprising one or more of the foregoing, wherein (xv) the phototherapeutic module comprises a light emitting diode (LED), or WOLED operably coupled on a carrying frame, configured to emit electromagnetic radiation through the lens at a selectable, frequency of between about 600 nm and about 900 nm with an intensity of about 2.5 and about 10 Candela at a distance of between about 2.0 cm and about 25 cm from the lens, the carrying frame operably coupled to the housing cap and wherein the light emitting diode is configured to selectively operate as a strobe having a variable strobe rate of between 1.0 and 60 Hz, (xvi) the negative ion generator is configured to generate negative ions at a density of between about 1×10⁵ negative ions/cm³ and about 3×10⁶ negative ions/cm³ at a distance of between about 2.0 cm and about 25 cm from the slits in the housing cap, wherein (xvii) the ozone module is configured to generate between about 11 and about 70 g/cm3 of ozone at a distance of between about 2.0 cm and about 25 cm from the slits in the housing cap, wherein (xviii) the spreading cap is transparent and or disposable, and (xix) is made of clear thermoplastic material or glass and is disposable.

While particular embodiments have been described, alternatives, modifications, variations, improvements, and substantial equivalents that are or may be presently unforeseen may arise to applicants or others skilled in the art. Accordingly, the appended claims as filed and as they may be amended, are intended to embrace all such alternatives, modifications variations, improvements, and substantial equivalents.

What is claimed is:

1. A portable device for treating a mood disorder, a headache or their combination in a subject in need thereof, the device comprising:
a. a substantially elongated housing, the housing comprising:
i. a dorsal housing portion and a ventral housing portion, each housing portion having a proximal end and a distal end;
ii. an air inlet opening disposed in each of the dorsal housing portion and the ventral housing portion;
iii. at least one air outlet slot disposed in the ventral housing portion and located between the ventral air inlet opening and the distal end of the ventral portion;
iv. a housing cap, operably coupled to the distal end of each of the dorsal housing portion and the ventral housing portion, the housing cap comprising an interchangeable lens and defines a plurality of arcuate slits in the periphery of the lens, wherein the dorsal housing portion is operably coupled to the ventral housing portion so as to define at least one compartment between the dorsal housing portion and the ventral housing portion; and
v. a display panel coupled to the dorsal housing portion;
b. a power supply;
c. a fan, configured to regulate reach of ozone, vibration, and negative ions.
d. a negative ion generating module;
e. a phototherapeutic module;
f. a vibration generator;
g. a control panel; and
h. optionally an ozone module, wherein the negative ion module, phototherapeutic module, the vibration generator and optionally the ozone generator are each in electronic communication with the power supply and the control panel, and wherein the device, using the control panel, is configured to selectively release negative ions through the slits disposed in the housing cap and/or the at least one air outlet slot in the ventral housing portion, and/or vibrate the housing cap, and/or emit phototherapeutic electromagnetic radiation through the lens.

2. The device of claim 1, wherein the phototherapeutic module comprises a light emitting diode (LED) operably coupled on a carrying frame, configured to emit electromagnetic radiation through the lens at a selectable, predetermined wavelength, duration and intensity, the carrying frame operably coupled to the housing cap.

3. The device of claim 2, wherein the selectable predetermined wavelength is between about 600 nm and about 900 nm with an intensity of about 2.5 and about 10 Candela at a distance of between about 2.5 cm and about 25 cm from the lens.

4. The method of claim 3, wherein the light emitting diode is configured to selectively operate as a strobe having a variable strobe rate of between 1.0 and 60 Hz.

5. The device of claim 1 wherein the negative ion module comprises:
a. a driver with a driver body and a drive shaft, in electronic communication with the power supply and the control panel;
b. a fan operably coupled to the motor's drive shaft, the fan disposed distally to the air inlet openings defined in each of the dorsal housing portion and ventral housing portion; and a negative ion generator comprising a drive circuit in electronic communication with the power supply;
c. a high voltage transformer operably coupled to the drive circuit, wherein the high voltage transformer is configured to amplify electromagnetic coupling via electromagnetic induction;
d. a rectifying circuit operably coupled to the high voltage transformer, configured to rectify the transformer output into a DC voltage; and
e. a plurality of electric discharge electrodes disposed proximally to the fan.

6. The device of claim 5, wherein the negative ion generator is configured to generate negative ions at a density of between about 1×10⁵ negative ions/cm³ and about 3×10⁶ negative ions/cm³ at a distance of between about 2.0 cm and about 25 cm from the slits in the housing cap.

7. The device of claim 5, wherein the vibration generator comprises a weighted cylinder selectively couplable to the drive shaft of the motor, the weighted cylinder being coupleable to the drive shaft between the fan and the motor body.

8. The device of claim 1, wherein the ozone module, when present, is configured to generate between about 11 and about 70 g/cm3 of ozone at a distance of between about 2.0 cm and about 25 cm from the slits in the housing cap.

9. The device of claim 1, wherein a spreading cap is operably coupled to the housing cap.

10. The device of claim 9, wherein the spreading cap comprises a receptacle; and slits configured to provide a spreading functionality to a cream, ointment, gel or a viscous liquid comprising one or more of the foregoing.

11. The device of claim 9, wherein the spreading cap is transparent and/or disposable.

12. The device of claim 1, further comprising a selectable AC-DC converter configured to provide DC voltage of between 1.0 and 10 Volts, the converter being in communication with a transformer port defined in the proximal end of the ventral housing portion.

13. A method of treating a mood disorder, a headache or their combination in a subject in need thereof, comprising:
a. providing a device comprising; a substantially elongated housing, the housing comprising: a dorsal housing portion and a ventral housing portion, each housing portion having a proximal end and a distal end; an air inlet opening disposed in each of the dorsal housing portion and the ventral housing portion; at least one air outlet slot disposed in the ventral housing portion and located between the ventral air inlet opening and the distal end of the ventral portion; a housing cap, operably coupled to the distal end of each of the dorsal housing portion and the ventral housing portion, the housing cap comprising a lens and defines a plurality of arcuate slits in the periphery of the lens, wherein the dorsal housing portion is operably coupled to the ventral housing portion so as to define at least one compartment between the dorsal housing portion and the ventral housing portion; and a display panel coupled to the dorsal housing portion; a power supply; a negative ion module; a phototherapeutic module; a vibration generator; a control panel; and an ozone module, wherein the negative ion module, phototherapeutic module, the vibration generator and the ozone generator are each in electronic communication with the power supply and the control panel, wherein the device, using the control panel, is configured to selectively release negative ions through the slits disposed in the housing cap and/or the at least one air outlet slot in the ventral housing portion, and/or vibrate the housing cap, and/or emit phototherapeutic electromagnetic radiation through the lens;
b. placing the distal end of the device abutting the forehead of the subject; and
c. using the control panel, selectively releasing negative ions through the slits disposed in the housing cap or the at least one air outlet slot in the ventral housing portion, vibrating the housing cap, emitting phototherapeutic electromagnetic radiation through the lens, generating ozone, or effecting any combination of at least three of the foregoing.

14. The method of claim 13, wherein the phototherapeutic module comprises a light emitting diode (LED) operably coupled on a carrying frame, configured to emit electromagnetic radiation through the lens at a selectable, frequency of between about 600 nm and about 900 nm with an intensity of about 2.5 and about 10 Candela at a distance of between about 2.5 cm and about 25 cm from the lens, the carrying frame operably coupled to the housing cap and wherein the light emitting diode is configured to selectively operate as a strobe having a variable strobe rate of between 1.0 and 60 Hz.

15. The method of claim 13, wherein the negative ion generator is configured to generate negative ions at a density of between about 1×10⁵ negative ions/cm³ and about 3×10⁶ negative ions/cm³ at a distance of between about 2.0 cm and about 25 cm from the slits in the housing cap.

16. The method of claim 13, wherein the ozone module, when present, is configured to generate between about 11 g/cm³ and about 70 g/cm³ of ozone at a distance of between about 2.0 cm and about 25 cm from the slits in the housing cap.

17. A method of increasing the permeability of at least a portion of the skin, comprising:
a. providing a device comprising; a substantially elongated housing, the housing comprising: a dorsal housing portion and a ventral housing portion, each housing portion having a proximal end and a distal end; an air inlet opening disposed in each of the dorsal housing portion and the ventral housing portion; at least one air outlet slot disposed in the ventral housing portion and located between the ventral air inlet opening and the distal end of the ventral portion; a housing cap, operably coupled to the distal end of each of the dorsal housing portion and the ventral housing portion, the housing cap comprising an interchangeable lens and a plurality of arcuate slits defined in the periphery of the lens; a spreading cap, operably coupled to the housing cap, configured to hold and spread a gel, an ointment, a cream or a viscous liquid comprising one or more of the foregoing, wherein the dorsal housing portion is operably coupled to the ventral housing portion so as to define at least one compartment between the dorsal housing portion and the ventral housing portion; a power supply; and a display panel coupled to the dorsal housing portion; a negative ion module; a phototherapeutic module; a vibration generator; a control panel; and an ozone module, wherein the negative ion module, phototherapeutic module, the vibration generator and the ozone generator are each in electronic communication with the power supply, the display panel and the control panel, wherein the device, using the control panel, is configured to selectively release negative ions through the slits disposed in the housing cap and/or the at least one air outlet slot in the ventral housing portion, and/or vibrate the housing cap, and/or emit phototherapeutic electromagnetic radiation through the lens;
b. contacting the skin portion where permeation of a cream, an ointment, a gel, or a viscous liquid is sought to be improved; and
c. using the control panel, selectively releasing negative ions through the slits disposed in the housing cap or the at least one air outlet slot in the ventral housing portion, vibrating the housing cap, emitting phototherapeutic electromagnetic radiation through the lens, generating ozone; and using the adaptor spreading the gel, ointment, cream or the viscous liquid comprising one or more of the foregoing.

18. The method of claim 17, wherein the phototherapeutic module comprises a light emitting diode (LED) operably coupled on a carrying frame, configured to emit electromagnetic radiation through the lens at a selectable, frequency of between about 600 nm and about 900 nm with an intensity of about 2.5 and about 10 Candela at a distance of between about 2.0 cm and about 25 cm from the lens, the carrying frame operably coupled to the housing cap and wherein the light emitting diode is configured to selectively operate as a strobe having a variable strobe rate of between 1.0 and 60 Hz.

19. The method of claim 17, wherein the negative ion generator is configured to generate negative ions at a density of between about 1×10⁵ negative ions/cm³ and about 3×10⁶ negative ions/cm³ at a distance of between about 2.0 cm and about 25 cm from the slits in the housing cap.

20. The method of claim 17, wherein the ozone module is configured to generate between about 11 and about 70 g/cm3 of ozone at a distance of between about 2.0 cm and about 25 cm from the slits in the housing cap.
